(19) United States
(12) Patent Application Publication
(10) Pub. No.: US 2023/0285638 A1
(43) Pub. Date: Sep. 14, 2023

(54) SILK FIBROIN COMPOSITE MATERIAL WITH INCREASED CONTENT OF BETA-SHEET AND METHOD FOR PREPARING THE SAME

(71) Applicant: Industry-Academic Cooperation Foundation, Yonsei University, Seoul (KR)

(72) Inventors: Jinkee HONG, Seoul (KR); Woojin CHOI, Seoul (KR); Moonhyun CHOI, Seoul (KR)

(21) Appl. No.: 18/177,659

(22) Filed: Mar. 2, 2023

(30) Foreign Application Priority Data
Mar. 4, 2022 (KR) 10-2022-0027838
Dec. 28, 2022 (KR) 10-2022-0187124

(51) Int. Cl. A61L 27/48; A61L 27/54

(52) U.S. Cl. CPC A61L 27/48; A61L 27/54; A61L 2300/204; A61L 2300/404

(57) ABSTRACT

The present invention relates to a silk fibroin composite material that overcomes the limitations of two physical properties, which were conventional trade-offs, by exhibiting excellent toughness and excellent ductility. Specifically, by preparing modified silk fibroin using a urethane oligomer as a casting substrate, a silk fibroin composite material exhibiting excellent toughness, tensile stress, and elastic modulus may be prepared.

### CROSS-REFERENCE TO RELATED APPLICATIONS

This application claims priority under 35 U.S.C. §119 to Korean Patent Application No. 10-2022-0027838, filed on Mar. 04, 2022, and Korean Patent Application No. 10-2022-0187124, filed on Dec. 28, 2022, in the Korean Intellectual Property Office, the disclosure of which is incorporated herein by reference in its entirety.

### TECHNICAL FIELD

The following disclosure relates to a silk fibroin composite material with increased content of a beta-sheet (β-sheet) and a method for preparing the same.

### BACKGROUND

Silk is the second most abundant natural material after cellulose. In general, silk refers to fibrous proteins released from Bombyx mori, and specifically, Bombyx mori threads released from cocoon floss are largely composed of two proteins: silk sericin and silk fibroin. Specifically, the cocoon threads contain about 75% fibroin protein, about 25% sericin protein, and about 3% minerals and carbohydrates. In order to expand the practicality of silk, pure fibroin proteins are separated through a degumming process that removes impurities and sericin with alkali, soap, enzyme, bleach, etc.

Silk fibroin material has excellent toughness among known protein materials and is used in various material industries. Specifically, the silk fibroin material is also used as a suture, a scaffold for cell therapy, etc. The physical properties described above may be explained by looking at a protein secondary structure of silk fibroin, and in particular, the more β-sheet crystals with strong internal attraction, the more resistant to the applied external force, and thus the higher tensile stress. However, the higher the β-sheet crystal content, the lower the amorphous content, which lowers the flexibility and makes the material easy to break, resulting in insufficient stretchability.

Therefore, it is necessary to research and develop silk fibroin that has excellent tensile stress according to high β-sheet crystal content and exhibits excellent ductility at the same time, overcoming the limitations of the trade-off between tensile stress and ductility.

### SUMMARY

An embodiment of the present invention is directed to providing a silk fibroin composite material with a high elastic modulus and toughness by overcoming the limitations of trade-off to exhibit excellent tensile stress and excellent ductility at the same time.

Another embodiment of the present invention is directed to providing a silk fibroin composite material having antimicrobial properties.

Yet another embodiment of the present invention is directed to providing a method for preparing a silk fibroin composite material having excellent maximum tensile stress, elastic modulus, and toughness by inducing crystallization of silk fibroin through a simple casting method to prepare modified silk fibroin.

Still another embodiment of the present invention is directed to providing a silk fibroin article such as a bioprosthetic manufactured using the silk fibroin composite material described above.

In order to achieve the object described above, the present inventors have continuously studied for the preparation of a silk fibroin composite material by overcoming the limitations of trade-off to exhibit excellent tensile stress and excellent ductility at the same time. As a result, when modified silk fibroin is prepared using a urethane oligomer as a casting substrate, it is surprisingly found that a silk fibroin composite material having excellent ultimate tensile stress, elastic modulus, and toughness may be prepared by greatly increasing a content of a β-sheet, thereby completing the present invention.

In one general aspect, there is provided a silk fibroin composite material comprising a modified silk fibroin that is modified from silk fibroin and has an increased content of a β-sheet, and having a peak showing a maximum intensity in the region of 1665 ± 10 cm⁻¹ of a spectrum according to Raman spectroscopy, a full width at half maximum of the peak being 10 to 80 cm⁻¹.

A ¹³C-NMR spectrum of the silk fibroin composite material may exhibit a first peak in the region of 169 ± 2 ppm and a second peak in the region of 172 ± 2 ppm.

The intensity of the second peak may be greater than the intensity of the first peak.

The ¹³C-NMR spectrum of the silk fibroin composite material may exhibit a third peak in the region of 22 ± 2 ppm.

The intensity of the third peak may be greater than the peak intensity in the region of 18 ± 2 ppm.

The natural silk fibroin may be derived from silkworm moths.

The silk fibroin composite material may comprise an antimicrobial urethane oligomer.

The antimicrobial urethane oligomer may be prepared by reacting a polyol with an antimicrobial isocyanate compound.

The polyol may be a polyether polyol having a weight average molecular weight of 100 to 1,000 g/mol.

The antimicrobial isocyanate compound may be a heterocyclic diisocyanate compound comprising a quaternary ammonium salt.

The polyol and the antimicrobial isocyanate compound may be comprised in a molar ratio of 1.3 to 5:1.

The antimicrobial urethane oligomer may have a hydroxyl group at its terminal.

The antimicrobial urethane oligomer may have a weight average molecular weight of 500 to 5,000 g/mol.

The silk fibroin composite material may satisfy Equation 1 below:

$$\left[\text{Equation1}\right] {\text{X}}_{1} / {\text{X}}_{2} > 1.5$$

wherein X₁ is a β-sheet content of modified silk fibroin calculated through Raman spectroscopy, and X₂ is a β-sheet content of natural silk fibroin.

The silk fibroin composite material may satisfy Equation 2 below:

$$\left[\text{Equation} 2\right] {\text{T}}_{1} / {\text{T}}_{2} > 4$$

wherein T₁ is a toughness of a silk fibroin composite material film, T₂ is a toughness of a natural silk fibroin film, and the toughness is measured according to ASTM D882 in a specimen having a size of 40 × 5.0 × 0.4 mm³.

The silk fibroin composite material may have a toughness of 300 MJ/m³ or more as measured according to ASTM D882 in a specimen having a size of 40 × 5.0 × 0.4 mm³.

In another general aspect, there is provided a silk fibroin article such as a bioprosthetic comprising the silk fibroin composite material as described above.

The article may be a bioprosthetic organ.

In another general aspect, there is provided a method for preparing a silk fibroin composite material, the method comprises:
A) manufacturing a urethane oligomer casting substrate;
B) casting a natural silk fibroin solution on the casting substrate; and
C) preparing a modified silk fibroin by drying the casting solution.

The (A) step may comprise: A-1) preparing a polymeric composition by mixing a polyol and an antimicrobial isocyanate compound; and A-2) casting the polymeric composition in a mold and then reacting the composition.

In the step (B), the natural silk fibroin solution may be a solution obtained by dissolving a natural silk fibroin protein and a kosmotropic salt in an acidic solvent.

The kosmotropic salt may be a combination of an alkali metal ion or an alkaline earth metal ion; and any one or two or more ions selected from the group consisting of sulfate (SO₄²⁻), phosphate (HPO₄²⁻), acetate (CH₃COO⁻), hydroxide (OH⁻), chloride (Cl⁻), bromide (Br⁻), formate (HCOO⁻), etc.

In the step (C), the drying may be performed at room temperature, and during drying, a natural silk fibroin protein comprised in the natural silk fibroin solution may be crystallized to prepare modified silk fibroin.

The method may further comprise, after the step (C), (D) detaching the modified silk fibroin from the casting substrate.

### BRIEF DESCRIPTION OF THE DRAWINGS

FIG. 1A is a spectrum according to Raman spectroscopy according to Examples 1 to 4 and Comparative Example 2, and FIG. 1B is a graph illustrating a β-sheet content calculated based on the spectrum.

FIG. 2 is a spectrum according to 400 MHz solid-state nuclear magnetic resonance (¹³C-NMR) according to Examples 1 to 4 and Comparative Example 2.

FIG. 3 illustrates the strain-stress curves of Examples 1 to 4 and Comparative Examples 2 and 3.

FIG. 4 is a graph illustrating the values of Equation 2 in Examples 1 to 4 and Comparative Examples 2 and 3.

FIG. 5 is a process for manufacturing a bioprosthetic valve from the silk fibroin composite material (IUB) according to Example 1 and images of the manufactured bioprosthetic valve.

FIG. 6 is a graph in which the performance of a bioprosthetic valve according to an embodiment is evaluated through Pulse Duplicator Hydrodynamic Testing, which is a condition similar to a heartbeat.

### DETAILED DESCRIPTION OF EMBODIMENTS

Hereinafter, a silk fibroin composite material according to the present invention and a method for preparing the same will be described in detail Here, unless otherwise defined, all technical terms and scientific terms have meanings commonly understood by those of ordinary skill in the art to which this invention belongs, and terms used in the description of the present invention are only for effectively describing specific embodiments and are not intended to limit the present invention.

In addition, descriptions of known effects and configurations that may unnecessarily obscure the subject matter of the present invention will be omitted in the following description. Hereinafter, units used in the specification without special mention are by weight, and as an example, the unit of % or ratio means % by weight or a ratio by weight, respectively.

In addition, terms “first”, “second”, A, B, (a), (b), and the like, will be used in describing components of the present invention. These terms are used only in order to distinguish any component from other components, and features, sequences, or the like, of corresponding components are not limited by these terms.

In addition, singular forms used in the specification of the present invention are intended to comprise the plural forms as well unless otherwise indicated in context.

Unless explicitly described otherwise, “including” or “comprising” any component in the specification will be understood to imply the inclusion of other components rather than the exclusion of other components.

In addition, numerical ranges used herein may include a lower limit, an upper limit, all values within that range, increments that are logically derived from the type and width of the defined range, all double-defined values, and all possible combinations of upper and lower limits of numerical ranges defined in different forms. Unless otherwise defined herein, values outside the numerical range that may arise due to experimental errors or rounded values are also included in the defined numerical range.

In addition, the term “oligomer” used herein may refer to a polymer having a weight average molecular weight of 100 to 10,000 g/mol.

Further, the term “full width at half maximum (FWHM)” used herein is a term indicating a width of a certain function, and refers to a difference between two independent variable values that are half of a maximum value of the function.

Hereinafter, a silk fibroin composite material according to the present invention and a method for preparing the same will be described in detail.

According to an embodiment of the present invention, the silk fibroin composite material may be prepared from natural silk fibroin. The natural silk fibroin may be derived from the silkworm moth, and in general, silk refers to a fibrous protein released from Bombyx mori. However, in addition to the above-mentioned, wild silk obtained from wild cocoons, such as hawthorn eating oak leaves, spider silk, which is a spider silk protein secreted by spiders, and seaweed silk secreted when seaweed adheres to rocks, are also called silk in a broad sense. However, considering high productivity, it is more common to use a silk fibroin protein obtained by extraction from Bombyx mori.

The silk fibroin protein is a form in which a crystal structure is added to an amorphous matrix, and the crystal structure is formed by stacking β-sheets in a layered form by a hydrogen bond. In the representative base sequences of silk fibroin extracted from Bombyx mori, GAGAGS accounts for about 53% and GAGAGY accounts for about 18%. Such a large portion is occupied by hydrophobic amino acids such as glycine (G) and alanine (A).

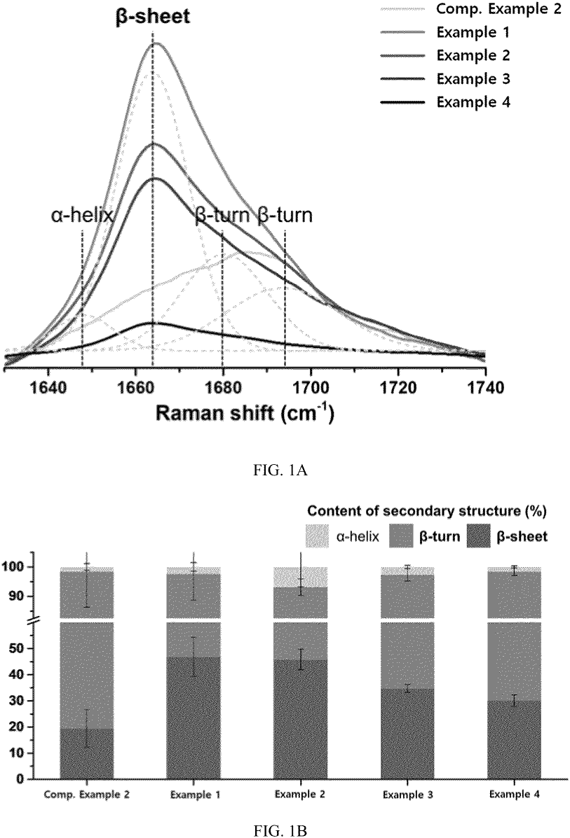

In one general aspect, there is provided a silk fibroin composite material comprising a modified silk fibroin that is modified from natural silk fibroin and has an increased content of a β-sheet, and having a peak showing a maximum intensity in a region of 1665 ± 10 cm⁻¹ of a spectrum according to Raman spectroscopy, a full width at half maximum of the peak being 10 to 80 cm⁻¹, as illustrated in FIG. 1A.

According to an embodiment of the present invention, as illustrated in FIG. 1A, the silk fibroin composite material according to an embodiment has a peak showing maximum intensity in the region of 1665 ± 10 cm⁻¹ of the spectrum according to Raman spectroscopy, and the peak may have a full width at half maximum of 10 to 80 cm⁻¹, specifically 15 to 60 cm⁻ ¹, and more specifically 20 to 50 cm⁻¹, or 30 to 42 cm⁻¹. The peak in the region of 1665 ± 10 cm⁻¹ may mean a β-sheet formed by modification from natural silk fibroin.

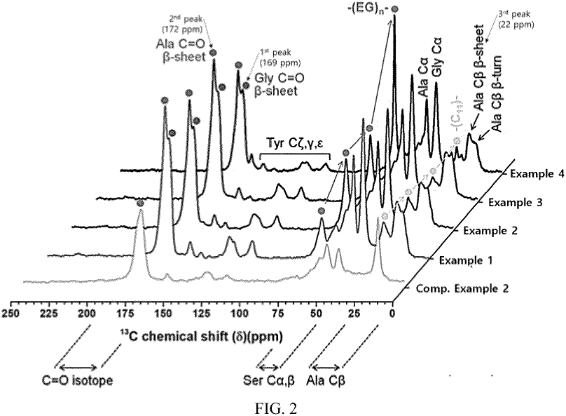

According to an embodiment of the present invention, as illustrated in FIG. 2, the ¹³C-NMR spectrum of the silk fibroin composite material may exhibit a first peak in the region of 169 ± 2 ppm and a second peak in the region of 172 ± 2 ppm, and specifically, the intensity of the second peak may be greater than the intensity of the first peak. In Examples 1 to 4, the β-sheet of glycine (Gly, C═O) was observed, which was not present in Comparative Example 2, and it may be verified that the β-sheet content increased in Examples than Comparative Example 2. Here, the first peak may refer to a β-sheet containing glycine (Gly) as a main amino acid, and the second peak may refer to a β-sheet containing alanine (Ala) as a main amino acid.

According to an embodiment of the present invention, as illustrated in FIG. 2, the ¹³C-NMR spectrum of the silk fibroin composite material may exhibit a third peak in the region of 22 ± 2 ppm. Specifically, the third peak may refer to a β-sheet containing alanine (Ala) as a major amino acid, and a peak in the region of 18 ± 2 ppm may refer to a β-turn structure. In the silk fibroin complex according to an embodiment of the present invention, since the β-sheet of alanine (Ala) is significantly more present than the β-turn structure of alanine, the intensity of the third peak may be greater than the peak intensity of the region of 18 ± 2 ppm. As illustrated in FIG. 2, although the ¹³C-NMR spectrum of Examples 1 to 4 exhibits a third peak in the region of 22 ± 2 ppm, the ¹³C-NMR spectrum of Comparative Example 2 does not exhibit a third peak, and a peak was observed in the region of 18±2 ppm instead of the region of 22 ± 2 ppm.

According to an embodiment of the present invention, the silk fibroin composite material may comprise an antimicrobial urethane oligomer. The antimicrobial urethane oligomer may be prepared by reacting a polyol with an antimicrobial isocyanate compound.

The polyol may be a polyol having a weight average molecular weight of 50 to 2,000 g/mol, specifically 100 to 1,000 g/mol, and more specifically 150 to 700 g/mol, and specifically may be a polyether polyol. Non-limiting examples of the polyether polyol comprise polyether polyols obtained by addition polymerization of alkylene oxides such as ethylene oxide, propylene oxide, and butylene oxide using water, low molecular weight polyol (propylene glycol, ethylene glycol, glycerin, trimethylolpropane, pentaerythritol, etc.), bisphenols (bisphenol A, etc.), dihydroxybenzene (catechol, resorcin, hydroquinone, etc.) or the like as an initiator. Specifically, polyethylene glycol, polypropylene glycol, polytetramethylene glycol, etc. are exemplified.

According to an embodiment of the present invention, the antimicrobial isocyanate compound may be used without significant limitation as long as it is a diisocyanate compound comprising an antimicrobial compound, and specifically may be a heterocyclic diisocyanate compound comprising a quaternary ammonium salt. The heterocyclic diisocyanate may be a compound derived from a trimer in which hexamethylene diisocyanate is bonded to isocyanurate (HMDI trimer), but the present invention is not limited thereto.

According to an embodiment of the present invention, the polyol and the antimicrobial isocyanate compound may be comprised in a molar ratio of 1.1 to 10:1, specifically 1.3 to 5:1, and more specifically 1.5 to 3:1. As the polyol is added in excess, a molecular weight may be controlled, and the antimicrobial urethane oligomer may also have a hydroxyl group at its terminal.

According to an embodiment of the present invention, the antimicrobial urethane oligomer may have a weight average molecular weight of 200 to 10,000 g/mol, specifically 500 to 5,000 g/mol, and more specifically 1,000 to 3,000 g/mol.

The silk fibroin composite material according to the present invention may implement significantly improved mechanical properties than conventional ones by preparing modified silk fibroin with an increased content of β-sheet by inducing modification of the natural silk fibroin using the above-described urethane oligomer as a casting substrate.

According to an embodiment of the present invention, as illustrated in FIG. 2, a ¹³C-NMR spectrum of the silk fibroin composite material may exhibit a fourth peak in the region of 30 ± 2 ppm and a fifth peak in the region of 70 ± 2 ppm. Specifically, the fourth peak may refer to an antimicrobial diisocyanate compound-derived unit of the antimicrobial urethane oligomer, and the fourth peak may mean a polyol-derived unit of the antimicrobial urethane oligomer. As the ¹³C-NMR spectrum of the silk fibroin composite material exhibits a fourth peak in the region of 30 ± 2 ppm and a fifth peak in the region of 70 ± 2 ppm, the existence of the antimicrobial urethane oligomer may be confirmed.

According to an embodiment of the present invention, the silk fibroin composite material may satisfy the following Equation 1, specifically Equation 3, and more specifically Equation 5:

$${\text{X}}_{1} / {\text{X}}_{2} > 1.5 \qquad \text{[Equation1]}$$

$${\text{X}}_{1} / {\text{X}}_{2} > 2.0 \qquad \text{[Equation 3]}$$

$${\text{X}}_{1} / {\text{X}}_{2} > 2.5 \qquad \text{[Equation 5]}$$

wherein X₁ is a β-sheet content of modified silk fibroin calculated through Raman spectroscopy, and X₂ is a β-sheet content of natural silk fibroin.

The silk fibroin composite material satisfying Equation 1, preferably Equation 3, and more preferably Equation 5 may exhibit a high content of β-sheet by having modified silk fibroin, and may implement significantly improved mechanical properties compared to natural silk fibroin before modification.

According to an embodiment of the present invention, the silk fibroin composite material may have a β-sheet content calculated by Raman spectroscopy of 20 to 80%, or 30% or more, specifically 40% or more, and more specifically 45% or more, or 45% to 80%. The silk fibroin composite material according to the present invention is prepared through a simple preparing method, and may exhibit significantly improved β-sheet content and high crystallinity, and also may implement excellent mechanical properties such as toughness, tensile stress, and elastic modulus.

According to an embodiment of the present invention, the silk fibroin composite material may satisfy the following Equation 2, specifically Equation 4, and more specifically Equation 6:

$$\left[\text{Equation} 2\right] {\text{T}}_{1} / {\text{T}}_{2} > 4$$

$$\left[\text{Equation} 4\right] {\text{T}}_{1} / {\text{T}}_{2} > 8$$

$$\left[\text{Equation} 6\right] {\text{T}}_{1} / {\text{T}}_{2} > 10$$

wherein T₁ is a toughness of a silk fibroin composite material film, T₂ is a toughness of a natural silk fibroin film, and the toughness is measured according to ASTM D882 on a specimen having a size of 40 × 5.0 × 0.4 mm³.

The silk fibroin composite material satisfying Equation 2, preferably Equation 4, and more preferably Equation 6 may exhibit excellent toughness, and may implement significantly improved mechanical properties compared to natural silk fibroin that does not contain modified silk fibroin.

Specifically, the silk fibroin composite material may have a toughness (T) of 300 MJ/m³ or more, specifically 400 MJ/m³ or more, or 500 MJ/m³ or more, and more specifically 500 to 3,000 MJ/m³ as measured according to ASTM D882 in a specimen having a size of 40 × 5.0 × 0.4 mm³.

Specifically, the silk fibroin composite material may have a toughness of 10 MPa or more, specifically 30 MPa or more, and more specifically 30 to 500 MPa, or 60 MPa or more as measured according to ASTM D882.

In addition, according to an embodiment of the present invention, the silk fibroin composite material may have a maximum tensile stress at break of 0.5 to 20 MPa, specifically 5 MPa or more, and more specifically 6 MPa or more, or 8 MPa or more as measured according to ASTM D882 in a specimen having a size of 40 × 5.0 × 0.4 mm³, and at the same time a strain at break of 30 to 400%, specifically 50% or more, and more specifically 70% or more.

The silk fibroin composite material according to an embodiment of the present invention satisfies all of the above-described toughness, tensile stress, and elastic modulus, and at the same time improves both tensile stress and ductility, which were conventional trade-offs, thereby overcoming the limitations between the two properties and exhibiting more significantly improved mechanical properties.

The present invention may provide a silk fibroin article such as a bioprosthetic comprising the silk fibroin composite material described above. The article may be one selected from the group consisting of films, sheets, gels or hydrogels, meshes, mats, nonwoven mats, fabrics, scaffolds, tubes, blocks, fibers, particles, powders, implants, foams, needles, and lyophilized articles, but the present invention is not limited thereto. In addition, even when the silk fibroin composite material described above is exposed to water, an internal hydrogen bond is maintained according to a high crystallinity, such that stickiness between materials is not induced. Therefore, the article may be suitable for use as a biomedical material because the shape and feeling of use are not deteriorated even when inserted into the body.

In addition, the article described above may be a technical textile. Here, the technical textile refers to a textile product that has a related additional function rather than a function originally required, and may be understood as medical fiber, industrial fiber, special fiber, high-performance fiber, or engineering fiber, and may mean a fiber having special functions such as biocompatibility, flame retardancy, conductivity, warmth retention, antimicrobial property, deodorizing effect, waterproofing, windproofing, and UV blocking. The technical textile according to the present invention may be used for sports and leisure textiles, medical and healthcare textiles, environmental textiles, or digital textiles based on excellent ductility and toughness.

In addition, the article described above may be a bioprosthetic organ. Examples of the bioprosthetic organs comprise, but are not limited to, organs, valves, blood vessels, joints, hearts, kidneys, and cartilage. Since the silk fibroin composite material according to an embodiment is not sticky even when exposed to water, has good body compatibility, and has excellent mechanical properties such as toughness, it may be widely used in the biomedical industry that may replace soft organs.

Hereinafter, a method for preparing a silk fibroin composite material according to an embodiment of the present invention will be described in more detail.

The present invention comprises a method for preparing a silk fibroin composite material, the method comprises:
A) manufacturing a urethane oligomer casting substrate;
B) casting a natural silk fibroin solution on the casting substrate; and
C) preparing a modified silk fibroin by drying the casting solution.

According to an embodiment of the present invention, the (A) step may comprise: A-1) preparing a polymeric composition by mixing a polyol; and an isocyanate compound or antimicrobial isocyanate compound; and A-2) casting the polymeric composition in a mold and then reacting the composition.

As the isocyanate compound, any cyclic diisocyanate compound having two or more isocyanate groups may be used without any particular limitation.

The antimicrobial isocyanate compound may be prepared by substituting an antimicrobial compound for an isocyanate compound, for example, by reacting a reactive compound comprising a quaternary ammonium salt with an isocyanate compound to finally prepare an antimicrobial isocyanate compound comprising a quaternary ammonium salt, but the present invention is not limited thereto. Specifically, the isocyanate compound may be a heterocyclic triisocyanate compound, and more specifically, the heterocyclic triisocyanate may be a trimer in which hexamethylene diisocyanate is bonded to isocyanurate (HMDI trimer), but the present invention is not limited thereto. In addition, the reactive compound comprising the quaternary ammonium salt may comprise a functional group such as amine or hydroxyl at its terminal, and an antimicrobial isocyanate compound comprising a quaternary ammonium salt may be prepared by reacting the functional group with the isocyanate of the isocyanate compound described above.

Examples of specific compounds of the polyol are the same as those described above, so the examples are omitted.

After mixing the polyol and the antimicrobial isocyanate compound and stirring the polymerizable composition with a mixer, casting the polymeric composition into a mold having a specific shape and reacting the composition may be performed. The reaction may be applied without any particular limitation as long as it is a conventional and known condition for the urethane reaction, and may be reacted at a temperature of 30° C. or more, and specifically 50° C. or more, for 5 hours, and specifically 10 hours or more. Furthermore, a drying step may be further performed, and drying may be performed at a temperature of 70° C. or more for 1 hour or more, but the present invention is not limited thereto.

According to an embodiment of the present invention, in the step (B), the natural silk fibroin solution may be a solution of a natural silk fibroin protein and a kosmotropic salt degummed in an acidic solvent. Specifically, the natural silk fibroin protein may be comprised in an amount of 0.1 to 50% (w/v), and specifically 0.5 to 30% (w/v) relative to the acidic solvent, and the kosmotropic salt may be comprised in an amount of 0.01 to 20% (w/v), and specifically 0.1 to 10% (w/v) relative to the acidic solvent.

The acidic solvent may be a C₁₋₇ organic acid. Examples of the acidic solvent comprise, but are not limited to, formic acid, acetic acid, propionic acid, butyric acid, lactic acid, fumaric acid, malic acid, citric acid, or a mixed solvent thereof.

The kosmotropic salt is known to contribute to stabilization of water molecules in an aqueous environment and also to the stabilization of intramolecular interactions in macromolecules such as proteins. The kosmotropic salt may be composed of a combination of an alkali metal ion or an alkaline earth metal ion; and any one or two or more ions selected from the group consisting of sulfate (SO₄²⁻), phosphate (HPO₄²⁻), acetate (CH₃COO⁻), hydroxide (OH⁻), chloride (Cl⁻), bromide (Br⁻), formate (HCOO⁻), etc. Preferably, the kosmotropic salt may be any one or two or more selected from calcium chloride, lithium bromide, potassium chloride, sodium chloride, and sodium acetate, and preferably calcium chloride, but the present invention is not limited thereto.

According to an embodiment of the present invention, the step (B) is performed by preparing the natural silk fibroin solution described above and casting the resulting solution on the casting substrate, and the shape of the casting substrate may vary depending on the shape to be manufactured, and accordingly, the amount of application of the solution to be cast may also vary.

Then, in the step (C), the drying may be performed at room temperature for 12 hours or more, and preferably 24 hours or more, and during drying, a natural silk fibroin protein comprised in the natural silk fibroin solution may be crystallized to prepare modified silk fibroin. Specifically, the modified silk fibroin may have a significantly improved content of β-sheet than the natural silk fibroin protein due to the influence of the urethane oligomer casting substrate described above during the crystallization process, and through this, a silk fibroin composite material comprising modified silk fibroin having a high crystallinity may be prepared.

The method may further comprise, after the step (C), (D) detaching the modified silk fibroin from the casting substrate. Through this, a silk fibroin composite material may be finally prepared. In addition, the prepared silk fibroin composite material may comprise the urethane oligomer described above.

Hereinafter, the silk fibroin composite material according to the present invention and a method for preparing the same will be described in more detail through examples. The following Examples are only a reference for describing the present invention in detail, and the present invention is not limited thereto and may be implemented in various forms.

### Physical Property Evaluation Method

1. Molecular weight: weight average molecular weight (Mw), number average molecular weight (Mn), and polydispersity index (PDI) in terms of standard polystyrene were obtained by gel permeation chromatography (GPC) measurement using chloroform as a solvent and equipped with a refractive index detector. A GPC equipment was ACQUITY APC from Waters, the column was ACQUITY APCTM from Waters, a column temperature was set to 30° C., an injection amount was 50 µl, and a flow rate was measured under a condition of 0.62 ml/min.

2. β-sheet content: a region from 1630 to 1750 cm⁻¹ in the spectrum according to Raman spectroscopy based on a 532 nm laser light source was analyzed. In the spectrum, the peaks in the regions of 1648 ± 2 cm⁻¹, 1665 ± 10 cm⁻¹ 1680 ± 2 cm⁻¹, and 1695 ± 2 cm⁻¹ represent α-helix, β-sheet, β-turn and β-turn structures, respectively, and as illustrated in FIG. 1A, each peak was classified in the form of a Gaussian function, and the β-sheet content was calculated using a peak intensity of the region of 1665 cm⁻¹ corresponding to the β-sheet content. Specifically, the β-sheet content was obtained by calculating the ratio of the peak area of the 1665 cm⁻¹ region relative to the total peak area.

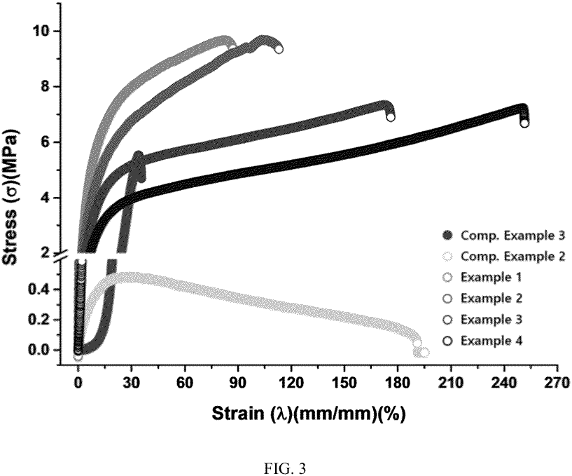

3. Maximum tensile stress (σₘₐₓ), strain (λ), toughness (T), and elastic modulus (E): in accordance with ASTM D882, UTM (Model 3366, Instron, USA) was used to measure a strain [mm/mm, %] according to the stress (σ) [MPa] under a tensile speed condition of 5.0 mm/min, and the measured strain-stress curves are shown in FIG. 3. The maximum tensile stress at break (σₘₐₓ) [MPa], the elastic modulus (E) [MPa] meaning the initial slope of the curve, and the toughness (T) [MJ/m³] meaning the area of the curve were obtained and listed in Table 2 below.

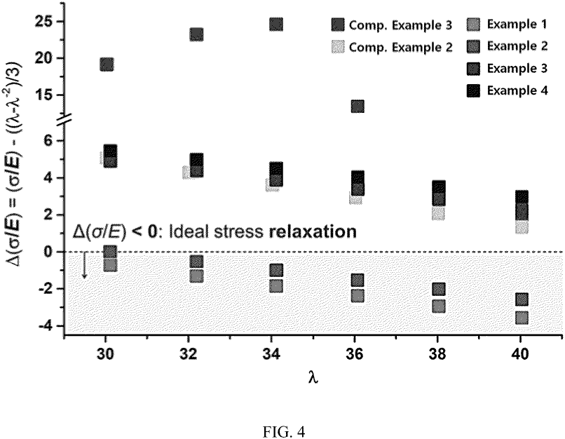

Furthermore, a stress relaxation efficiency was predicted based on Equations 1 and 2 below, and the value of Equation 2 was graphed and illustrated in FIG. 4. Specifically, Equation 1 was based on a Neo-Hookean solid model. Equation 2 calculated the difference by subtracting the value of Equation 1 of the Neo-Hookean solid model from the value of the silk fibroin composite material according to the embodiment, and when the calculated value (Δ(σ/E)) is negative (Δ(σ/E) < 0), it was determined that the article exhibits an ideal stress relaxation behavior and is suitable for use as a bioprosthetic organ (see, Science, 372, 1078-1081 (2021)).

$$\frac{σ}{E} = \left(λ - {λ}^{- 2}\right) / 3 \qquad \text{[Equation 1]}$$

$$Δ \left(\frac{σ}{E}\right) = \left(\frac{σ}{E}\right) - \left[\left(λ - {λ}^{- 2}\right) / 3\right] \qquad \text{[Equation 2]}$$

wherein σ is a stress, λ is a strain, and E is an elastic modulus.

### [Preparation Example 1] Preparation of Natural Silk Fibroin

After cutting 5 g of Bombyx mori into a size of 1 cm, 4.24 g of sodium carbonate (Na₂CO₃) was dissolved in 2 ℓ of distilled water, soaked in an alkaline soap solution (0.02 M Na₂CO₃), heated to 85° C. or more and then degummed for 1 hour. Thereafter, after washing twice for 20 minutes with distilled water, drying was performed at room temperature to obtain 3.7 g of refined natural silk fibroin (yield: 75%).

### [Preparation Example 2] Manufacture of Urethane Oligomer Casting Substrate

After adding 0.979 g of N-(2-amino-2-oxoethyl)-N,N-dimethyldodecan-1-aminium chloride (Angenechemical, Hong Kong) to 10 mℓ of propylene carbonate (Sigma Aldrich, USA) and dissolving it at 70° C., 1.61 g of hexamethylene diisocyanate trimer (BLD Pharm, China) was added therein and reacted at 80° C. for 1 hour or more to obtain an antimicrobial diisocyanate compound.

Subsequently, polyethylene glycol (Mw: 200 g/mol) was added to the prepared antimicrobial diisocyanate compound and evenly stirred using a vortex mixer to prepare a polymeric composition. Here, the molar ratio of the polyethylene glycol to the antimicrobial diisocyanate compound was added to satisfy 2:1. The polymeric composition was cast into a 40 × 5.0 mm2 (length x width) beam-shaped mold, reacted and dried at 80° C. in a vacuum condition for 5 days to finally manufacture a urethane oligomer casting substrate. The weight average molecular weight of the prepared urethane oligomer was measured as 1690 g/mol.

### Preparation Examples 3 to 5

Preparation Examples 3 to 5 were performed in the same manner as in Preparation Example 2 except that polyethylene glycols having a weight average molecular weight (Mw) of 400 g/mol, 600 g/mol, and 1,000 g/mol, respectively, were used. Here, the molar ratio of the polyethylene glycol to the antimicrobial diisocyanate compound was added to satisfy 2:1.

### Examples 1 to 4

0.3 g of calcium chloride (CaCl₂) was mixed with 10 ml of formic acid, and the dispersion was sonicated for 1 hour to obtain a homogeneous solution. 1 g of the degummed silk fibroin of Preparation Example 1 was added to the solution and dissolved at 60° C. for 1 hour to obtain a natural silk fibroin solution.

The natural silk fibroin solution was cast on the urethane oligomer casting substrates of Preparation Examples 2 to 5, dried at room temperature for 3 days and detached from the casting substrate to finally obtain a silk fibroin composite material having a size of 40 × 5.0 × 0.4 mm³ according to Examples 1 to 4. The prepared silk fibroin composite material was analyzed by Raman spectroscopy and solid-state nuclear magnetic resonance (¹³C-nuclear magnetic resonance (¹³C-NMR)), and the results are illustrated in FIGS. 1A, 1B and 2.

Specifically, FIG. 1A is a spectrum according to the Raman spectroscopy, and FIG. 1B illustrates a graph illustrating β-sheet content measured based on the spectrum according to the Raman spectroscopy. In addition, a solid state ¹³C-NMR spectrum is illustrated in FIG. 2.

### Comparative Example 1

A natural silk fibroin film according to Comparative Example 1 was obtained by preparing the degummed natural silk fibroin of Preparation Example 1 into a specimen having a size of 40 × 5.0 × 0.4 mm³.

### Comparative Example 2

The natural silk fibroin solution of Example 1 was cast on a polystyrene substrate, dried at room temperature for 24 hours, and then the specimen was detached from the casting substrate to finally obtain a silk fibroin film according to Comparative Example 2 having a size of 40 × 5.0 × 0.4 mm³. The manufactured silk fibroin film was analyzed by Raman spectroscopy and ¹³C-NMR, and the results are illustrated in FIGS. 1A, 1B and 2.

TABLE 1

| | Example 1 | Example 2 | Example 3 | Example 4 | Comp. Example 1 | Comp. Example 2 |
|---|---|---|---|---|---|---|
| Urethane oligomer casting substrate | Preparatio n Example 2 | Preparatio n Example 3 | Preparati on Example 4 | Preparati on Example 5 | - | Polystyrene |
| Mw of polyethylene glycol [g/mol] | 200 | 400 | 600 | 1,000 | - | - |
| β-sheet content [%] | 46.8 | 45.8 | 34.7 | 30.2 | 16 | 19.4 |

As shown in Table 1, it was confirmed that the silk fibroin composite materials of Examples 1 to 4 had a significantly higher β-sheet content than Comparative Examples 1 and 2. In particular, Examples 1 and 2, in which the weight average molecular weight of polyethylene glycol was 100 to 500 g/mol, showed a higher β-sheet content.

It was confirmed from FIG. 1A that when the protein secondary structure was analyzed through Raman spectroscopy, Example 1 had a peak showing a maximum intensity in the region of 1663 cm⁻¹ corresponding to the β-sheet, the full width at half maximum of the peak was 40 cm⁻¹, and the intensity of the peak in the 1663 cm⁻¹ region was about 4 times higher than that of Comparative Example 2. In addition, Example 1 also had a peak showing the maximum intensity in the region of 1663 cm⁻¹, and the full width at half maximum of the peak was 32.5 cm⁻¹.

Furthermore, in the ¹³C-NMR spectrum according to the 400 MHz solid-state nuclear magnetic resonance method of FIG. 2, in the case of Examples 1 to 4, large peaks were observed in the region of 150 to 190 ppm (C = O) and the region of 5 to 25 ppm corresponding to the β-sheet of alanine (Ala) (Ala Cβ). Through this, it was confirmed that both glycine (Gly) and alanine (Ala) of Example 1 were crystallized. Furthermore, the presence of the antimicrobial urethane oligomer could be confirmed through the peaks of 30 ppm (-(C₁₁)-) and 70 ppm (-(EG)ₙ-) in the spectra of Examples 1 to 4.

Table 2 below shows the maximum tensile stress, the elastic modulus, and the toughness of Examples 1 to 4 and Comparative Example 2. In addition, as Comparative Example 3, the toughness of bovine pericardium (BP) used for the bioprosthetic valve material was also shown.

TABLE 2

| | Example 1 | Example 2 | Example 3 | Example 4 | Comp. Example 2 | Comp. Example 3 |
|---|---|---|---|---|---|---|
| σₘₐₓ[MPa] | 9.0 | 8.9 | 5.2 | 5.0 | 0.43 | 5.3 |
| E[MPa] | 86.2 | 67.6 | 34.7 | 25.6 | 3.2 | 29.9 |
| T[MJ/m³] | 764.0 | 818.5 | 1039.9 | 1311.6 | 65.2 | 79.3 |

In addition, it was confirmed that the tensile stress of Example 1 was increased by about 20.9 times, the elastic modulus was improved by about 27 times, and the toughness was improved by about 12 times or more compared to Comparative Example 2. Through this, it was confirmed that the silk fibroin composite material according to an embodiment of the present invention overcomes the limitations of toughness and ductility, which were conventional trade-offs, and thus exhibits excellent toughness and at the same time implements excellent ductility due to the increased content of the β-sheet.

In addition, the silk fibroin composite materials of Example 1 and Comparative Example 2 were immersed in water and the change was observed. In the case of Comparative Example 2, when the silk fibroin composite materials were immersed in water and then placed on top of each other, they became sticky and were not easily separated, but in the case of Example 1, hydrogen bonds were not broken and were easily separated even when exposed to water. Through this, it could be confirmed that the silk fibroin composite material according to an embodiment of the present invention with a high content of the β-sheet, exhibits non-stick properties even when exposed to water without breaking hydrogen bonds, and may be easily used in an internal environment where fluid such as blood is always present, and by using this characteristic, it is possible to use it in the medical industry, such as a biomedical material that may be inserted into the body.

### [Evaluation Example] Bioprosthetic Organ Manufacturing and Performance Evaluation

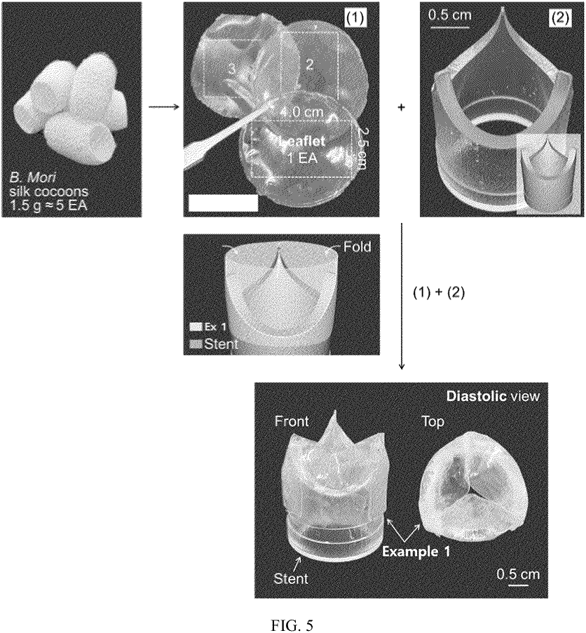

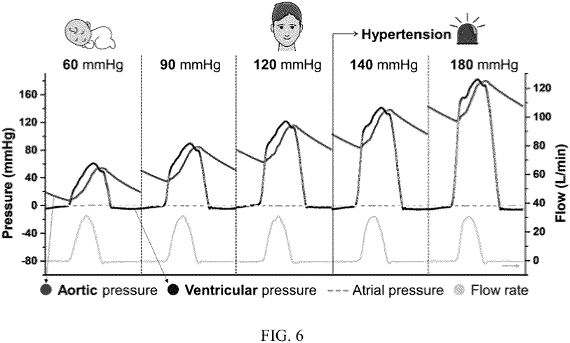

A bioprosthetic valve was manufactured using a stent and three silk fibroin composites (IUB) according to Example 1, and a manufacturing process and an image of the bioprosthetic valve are illustrated in FIG. 5. Furthermore, the performance of the bioprosthetic valve was evaluated through Pulse Duplicator Hydrodynamic Testing, which is a condition similar to a heartbeat, and a graph of the results is illustrated in FIG. 6.

Specifically, FIG. 6 is a test result by applying the manufactured bioprosthetic valve, and illustrates aortic pressure, ventricular pressure, atrial pressure and flow rate under pressure conditions of 60 to 180 mmHg. It was confirmed from the graph of FIG. 6 that the bioprosthetic valve manufactured using the silk fibroin composite material according to an embodiment operated normally under various pressure conditions by maintaining a constant pulsatile flow rate of about 30 L/min under a pressure condition of 60 to 180 mmHg.

In addition, the bioprosthetic valve according to an embodiment satisfied all of the physical property standards according to ISO 5840-3 and showed physical properties equivalent to those of the above evaluation results even after 120 days. Furthermore, the β-sheet content of the silk fibroin composite material prepared by reusing the urethane oligomer casting substrate according to Preparation Example 2 was 47.7%, 51.8%, 50.4%, and 48.4%, respectively, showing a β-sheet content equivalent to the β-sheet content (48.8%) of Example 1, thereby confirming the recyclability of the urethane oligomer casting substrate.

Through this, the silk fibroin composite material according to the present invention has a high β-sheet content, and exhibits excellent maximum tensile stress, elastic modulus and toughness, and thus the conventional trade-off between toughness and ductility is complementarily improved. In addition, it was confirmed that when applied to the bioprosthetic organ using the silk fibroin composite material, the bioprosthetic organ showed excellent performance and may be used more widely in various forms in the biomedical field.

The silk fibroin composite material according to the present invention has excellent tensile stress and exhibits excellent ductility at the same time to overcome the limitations of the two physical properties that were conventionally contradictory. Specifically, the silk fibroin composite material according to the present invention may have significantly improved toughness, tensile stress, and elastic modulus compared to conventional silk fibroin, and thus may be applied to various articles. Furthermore, the silk fibroin composite material exhibits non-stick properties because hydrogen bonds are not broken even when exposed to water, so it may be easily used in an in vivo environment where fluid is always present, and may be widely used in biomedical industries such as bioprosthetic organs.

Although the preferred embodiments of the present invention have been described above, the present invention is not limited thereto, and it is possible to make various modifications and practice within the scope of the claims and the description and the accompanying drawings, and this is also natural to belong to the scope of the invention.

What is claimed is:

1. A silk fibroin composite material, comprising a modified silk fibroin that is modified from natural silk fibroin and has an increased content of a beta-sheet (β-sheet), and having a peak showing a maximum intensity in the region of 1665 ± 10 cm⁻¹ of a spectrum according to Raman spectroscopy, a full width at half maximum of the peak being 10 to 80 cm⁻¹.

2. The silk fibroin composite material of claim 1, wherein a ¹³C-NMR spectrum of the silk fibroin composite material exhibits a first peak in the region of 169 ± 2 ppm and a second peak in the region of 172 ± 2 ppm.

3. The silk fibroin composite material of claim 2, wherein the intensity of the second peak is greater than the intensity of the first peak.

4. The silk fibroin composite material of claim 1, wherein a ¹³C-NMR spectrum of the silk fibroin composite material exhibits a third peak in the region of 22 ± 2 ppm.

5. The silk fibroin composite material of claim 4, wherein the intensity of the third peak is greater than the peak intensity in the region of 18 ± 2 ppm.

6. The silk fibroin composite material of claim 1, wherein the silk fibroin composite material comprises an antimicrobial urethane oligomer.

7. The silk fibroin composite material of claim 6, wherein the antimicrobial urethane oligomer is prepared by reacting a polyol with an antimicrobial isocyanate compound.

8. The silk fibroin composite material of claim 7, wherein the polyol is a polyether polyol having a weight average molecular weight of 100 to 1,000 g/mol.

9. The silk fibroin composite material of claim 7, wherein the antimicrobial isocyanate compound is a heterocyclic diisocyanate compound comprising a quaternary ammonium salt.

10. The silk fibroin composite material of claim 7, wherein the polyol and the antimicrobial isocyanate compound is comprised in a molar ratio of 1.3 to 5:1.

11. The silk fibroin composite material of claim 6, wherein the antimicrobial urethane oligomer has a hydroxyl group at its terminal.

12. The silk fibroin composite material of claim 1, wherein the silk fibroin composite material satisfies Equation 1 below:
$${\text{X}}_{1} / {\text{X}}_{2} > 1.5 \qquad \text{[Equation 1]}$$
wherein X₁ is a β-sheet content of modified silk fibroin calculated through Raman spectroscopy, and X₂ is a β-sheet content of natural silk fibroin.

13. The silk fibroin composite material of claim 1, wherein the silk fibroin composite material satisfies Equation 2 below:
$${\text{T}}_{1} / {\text{T}}_{2} > 4 \qquad \text{[Equation 2]}$$
wherein T₁ is a toughness of a silk fibroin composite material film, T₂ is a toughness of a natural silk fibroin film, and the toughness is measured according to ASTM D882 in a specimen having a size of 40 × 5.0 × 0.4 mm³.

14. The silk fibroin composite material of claim 1, wherein the silk fibroin composite material has a toughness of 300 MJ/m³ or more as measured according to ASTM D882 in a specimen having a size of 40 × 5.0 × 0.4 mm³.

15. A bioprosthetic comprising the silk fibroin composite material of claim 1.

16. A method for preparing a silk fibroin composite material, the method comprising:
A) manufacturing a urethane oligomer casting substrate;
B) casting a natural silk fibroin solution on the casting substrate; and
C) preparing a modified silk fibroin by drying the casting solution.

17. The method of claim 16, wherein the (A) step comprises:
A-1) preparing a polymeric composition by mixing a polyol and an antimicrobial isocyanate compound; and
A-2) casting the polymeric composition in a mold and then reacting the composition.

18. The method of claim 16, wherein in the step (B), the natural silk fibroin solution is a solution obtained by dissolving a natural silk fibroin protein and a kosmotropic salt in an acidic solvent.

19. The method of claim 16, wherein the kosmotropic salt is a combination of an alkali metal ion or an alkaline earth metal ion; and any one or two or more ions selected from the group consisting of sulfate (SO₄²⁻), phosphate (HPO₄²⁻), acetate (CH₃COO⁻), hydroxide (OH⁻), chloride (Cl⁻), bromide (Br⁻), and formate (HCOO⁻).

20. The method of claim 16, wherein in the step (C), the drying is performed at room temperature, and during drying, a natural silk fibroin protein comprised in the natural silk fibroin solution is crystallized to prepare modified silk fibroin.
